SEQUENCE LISTING (1 sequence)

SEQ ID NO: 1 - DNA, 495 bp, Artificial Sequence
  recombinant sequence for protein secretion
gccggcattggtcaagttgaagatgttgaatcagaatatcataaaacacttatgaaaccaccagaagaaaaagaaaaaatttcaaaagaaattcttaatggtaaagttccaattcttcttcaagctatttgtgaaacacttaaagaatcaacaggtaatttgacagttggtgataaagttacacttgctgatgttgttcttattgcttcaattgatcatattacagatcttgataaagaatttttgacaggtaaatatccagaaattcataaacatcgtaaacatcttttggctacatcaccaaaacttgctaaatatctttcagaacgtcatgctacagcttttttttccatcagagacagtgcacacaggcggtttctgctattccggagagcattcaaacagttggacgtagaagcagctctgaccaaagcccttggggaagtggacattcttctgacctggatgcagaaattctacaagctctaagccggc

(19) United States
(12) Patent Application Publication
(10) Pub. No.: US 2014/0341853 A1
(43) Pub. Date: Nov. 20, 2014

(54) Bacteria-Mediated Therapy for Cancer

(71) Applicant: Vanna Hovanky, Austin, TX (US)

(72) Inventor: Vanna Hovanky, Austin, TX (US)

(21) Appl. No.: 14/276,274

(22) Filed: May 13, 2014

Related U.S. Application Data
(60) Provisional application No. 61/822,915, filed on May 14, 2013.
(60) Provisional application No. 61/877,313, filed on Sep. 13, 2013.

(51) Int. Cl. A61K 35/74; A61K 31/7052; A61K 31/513; A61K 31/522; A61K 31/7028

(52) U.S. Cl. CPC A61K 35/747; A61K 35/74; A61K 35/744; A61K 35/745; A61K 31/522; A61K 31/7028; A61K 31/513; A61K 31/7052
USPC 424/93.2

(57) ABSTRACT

Methods for treating tumors and malignant tumors in regions that are adjacent to the gastrointestinal tract are provided. Therapeutically effective amounts of transformed bacteria are administered to subjects in need of treatment. Bacteria are transformed to produce proteins exhibiting therapeutic effects. These therapeutic effects can be the production of an enzyme that catalyzes the conversion of a prodrug into a drug and/or a protein that has therapeutic activity on its own. Bacteria may be provided to the gastrointestinal tract of the subject in need of treatment or preventative measures. In some cases, a prodrug is additionally administered.

### CROSS REFERENCE TO RELATED APPLICATIONS

The present application claims priority to U.S. Provisional Patent Application No. 61/822,915, filed May 14, 2013, entitled “Bacteria-mediated gene therapy for cancer” and U.S. Provisional Patent Application No. 61/877,313, filed Sep. 13, 2013, entitled “Expression of cancer killing or cancer suppressing therapeutic proteins by bacteria to serve as active cultures in the manufacturing of food products or as a probiotic,” the disclosures of which are incorporated herein by reference.

### FIELD OF THE INVENTION

The present disclosure relates generally to bacterially mediated therapy, transformed bacteria, prodrugs, cancer treatments, and colon cancers.

### BACKGROUND INFORMATION

Colon cancer is the third most common cancer and the fourth most common cause of cancer deaths in the world. Most colon cancers begin as small benign polyps growing in the innermost layer of the large intestines, called the mucosa. While small polyps can be removed during colonoscopies, larger cancers must be removed by extensive surgeries. Chemotherapy is currently commonly used with surgeries. However, due to lack of target organ selectivity, chemotherapeutic drugs often cause side effects in other parts of the body, such as hair loss, low blood cell counts, and increased chance of infection.

### BRIEF DESCRIPTION OF THE FIGURES

Material described and illustrated is provided to exemplify aspects and is not meant to limit scope. For simplicity and clarity of illustration, elements illustrated in the figures are not necessarily drawn to scale. Further, where appropriate, reference labels have been repeated among figures to indicate corresponding or analogous elements. In the figures:

FIG. 1 schematically illustrates bacterially mediated prodrug cancer therapy.

FIGS. 2A and 2B illustrate results of studies in which growing yeast cells were exposed to transformed E. coli BL21 and the prodrug Daun02.

FIG. 3 illustrates some mechanisms of action for anthracycline molecules on tumor cells.

FIG. 4 shows a plasmid map for a plasmid containing the human IL-24 coding DNA sequence.

### DETAILED DESCRIPTION

In the following description, specific details are set forth in order to provide an understanding of certain embodiments. Embodiments may be practiced without one or more of these specific details and frequently specific details of one embodiment may be practiced with other disclosed embodiments, as will be apparent to one of skill in the art. In other instances, well-known features are not described in detail in order to not obscure the description of certain embodiments.

Methods useful for the treatment and prevention of tumors and malignant tumors (cancers) are provided. Embodiments are useful for treating tumors and malignant tumors in the gastrointestinal system. In some embodiments, treatment is directed to the large intestine. Types of tumors and malignant tumors include, for example, gastrointestinal cancer, colon cancer, rectum, colon carcinoma, colorectal adenoma, intrahepatic bile duct cancer, stomach cancer, gastric cancer, pelvic cancer, esophageal cancer, small intestine cancer, villous colon adenoma, and gastrointestinal carcinoid tumors. Bacteria are transformed to produce proteins exhibiting therapeutic effects. These therapeutic effects can be the production of an enzyme that catalyzes the conversion of a prodrug into a drug and/or a protein that exhibits therapeutic activity on its own. Therapeutically effective amounts of transformed bacteria are provided to subjects in need of treatment or preventative measures. In some embodiments, bacteria are provided to an intestine of the subject in need of treatment or preventative measures. In further embodiments, a prodrug is additionally administered to a subject in need of treatment or preventative measures.

A variety of anticancer proteins exist that exhibit tumor destroying or inhibiting effects, either alone or through catalytic action on a prodrug. A prodrug is a molecule or compound that enters the body as non- or minimally therapeutic substance and is capable of undergoing one or more chemical changes in vivo that transform the prodrug molecule or compound into a therapeutic molecule or compound. A prodrug is a precursor to the therapeutic compound or molecule.

An emerging approach for the treatment of cancer is called gene therapy, the delivery of genes or therapeutic proteins to affected tissue. Viruses have recently been researched for their use as gene delivery vehicles, or vectors, for gene therapy. However, viral-vector therapy can have drawbacks. Viruses carry a variety of safety concerns such as the potential for increased side effects, mutagenesis through insertion of viral DNA into human DNA in the wrong place, and the release of viral particles in the environment. Viral vector systems are more expensive, needing complex methods for cell culturing, special media, and proper storage (Wei et al., “Clostridial spores as live ‘Trojan horse’ vectors for cancer gene therapy: comparison with viral delivery systems,” Genetic Vaccines and Therapy, 6:8, 2008).

A solution to the problems of current gene therapy methods may be found in the use of bacteria. Circular DNA molecules, called plasmids, are present in many species of bacteria and are capable of being manipulated. Bacteria containing plasmids do not carry the potential to mutate host DNA (unlike viral DNA), and they can express full, functioning proteins. Additionally, bacteria such as E. coli have a low likelihood of rejection by the body and can be produced through relatively low cost cell culturing and media techniques.

The proximity of the colon (large intestine) mucosa layers where colon cancers form, to the aerobic bacteria colonizing there, such as E. coli, enables treatment through bacterial-vector therapy. In embodiments, bacteria are engineered to produce a protein having a therapeutic purpose, such as, for example, the ability to catalyze the conversion of a prodrug into a drug, or another positive immunological, anti-angiogenic, or other tumor suppressing ability. The DNA in the bacteria can also be engineered to be more tumor specific through the inclusion of a coding DNA sequence for a therapeutic protein that is tumor-targeting. A therapeutic protein can target a tumor through, for example, its interactions with other proteins in the cancer-related proteome. Additionally, bacteria can also be engineered to be more tumor specific through the selection of a bacterial species that has intrinsic tumor locating properties such as being inclined to colonize in anaerobic or hypoxic conditions, or through the use of a tumor specific promoter in an engineered DNA sequence.

Plasmids including one or more therapeutically useful genes are used to transform bacterial cells. The engineered bacteria then go through an incubation growth process. After having reached a stable growth rate they can be stored for later delivery or immediately encapsulated for delivery. For example, transformed bacteria can be delivered orally in pill, powder, or suspension form in a liquid, a timed-release oral dose, by adding to a food base such as, for example, a cultured milk product such as yogurt, a cultured foodstuff, a foodstuff, or rectally as a suppository or a combination thereof. Cultured foodstuffs include probiotic beverages that may be dairy or non-dairy, and can include, for example, fermented oat drinks, altered fruit juices, or milk/yogurt-based beverages. Foodstuffs include, for example, cereal bars and chocolate. Useful bacterial vectors include, for example, E. coli, and bacteria from the genera: Lactococcus, Streptococcus, Clostridium, Salmonella, Listeria, Prevotella, Bifidobacterium, Leuconostoc, Peseudomonas, and Lactobacillus species (some of the listed bacterial species refer to deactivated strains that are non-virulent). Other examples of suitable bacteria species include: Streptococcus thermophilus, Lactobacillus bulgaricus, and Bifidobacterium lactis (BB-12).

In some embodiments, the protein is β-galactosidase (beta-gal). Both human and E. coli versions of the gene-encoding for the beta-gal enzyme (lac gene) exist for the metabolism of lactose. However, native colon E. coli lac genes are maintained in an inactive state by the constant presence of a repressor protein that is only released in the presence of lactose.

E. coli beta-gal (non-human) can catalyze the prodrug Daun02 (a daunorubicin beta-galactoside prodrug: N-[4″-(β-galactopyranosyl)-3″-nitrobenzyloxycarbonyl]daunomycin) into a toxic drug (daunorubicin) that may be used to kill or inhibit cancer cells. Daun02 is a derivative of the anthracycline daunomycin. Anthracyclines are a class of drugs currently used to treat several types of cancer. Anthracyclines can cause cancer cell death by binding to proteasomes and cancer cell DNA. Anthracyclines an also interfere with topoisomerase function, causing accumulation of cancer cell DNA damage. Other useful prodrugs and enzymes that transform prodrugs into therapeutically active molecules include, for example, gal-DNC4 (N-[(4″R,S)-4″-ethoxy-4″-(1′″-O-β-D-galactopyranosyl)butyl]daunorubicin) (transformative enzymes include: beta-gal), nucleoside or amino acid analogs such as 5-fluorocytosine (transformative enzymes include: cytosine deaminase), polymerase inhibitors such as Poly-ADP (adenosine diphosphate ribose) ribose polymerase-1 inhibitors (transformative enzymes include: glutathione (GSH), glutathione S-transferase P1 (GSTP1)), CNOB (6-chloro-9-nitro-5-oxo-5H-benzo(a)phenoxazine) (transformative enzymes include: E. coli nitroreductase and its alternative form ChrR6), ganciclovir (transformative enzymes include: herpes simplex 1 virus thymidine kinase), nitrogen mustard 1 glutamates, such as CMDA (4 [(2-chloroethyl)(2-mesyloxyethyl)amino]benzoyl l-glutamic acid) (transformative enzymes include: carboxypeptidase G2), 6-methylpurine deoxyriboside (transformative enzymes include: purine nucleoside phosphorylase (PNP)), Irinotecan (CPT 11), (5-[aziridin-1-yl]-2,4-dinitrobenzamide) (CB1954) (transformative enzymes include: nitrogen reductase), doxorubicin prodrugs (transformative enzymes include: penicillin-V amidase), 5-fluorocytosine (transformative enzymes include: cytosine-deaminase (CD)), and cyclophosphamide (transformative enzymes include: cytochrome P450 (CYP450)). Bacteria are transformed with plasmids capable of expressing one or more enzymes that catalyze the conversion of a prodrug into an active drug form.

In some embodiments, a prodrug or a combination of prodrugs is administered to the subject after the engineered bacterium has been administered. In some embodiments, the prodrug is administered after the engineered bacterium has had time to colonize around the cancer cells. The prodrug(s) can be administered orally, in a therapeutic amount, in the form of, for example, a pill, a timed release capsule, in a foodstuff, a liquid solution or suspension, or as a powder, rectally as a suppository in solid or liquid form, injected into the patient in a solution or suspension, or a combination thereof. The therapeutic amount of the prodrug can be injected, for example into an artery leading to the cancer.

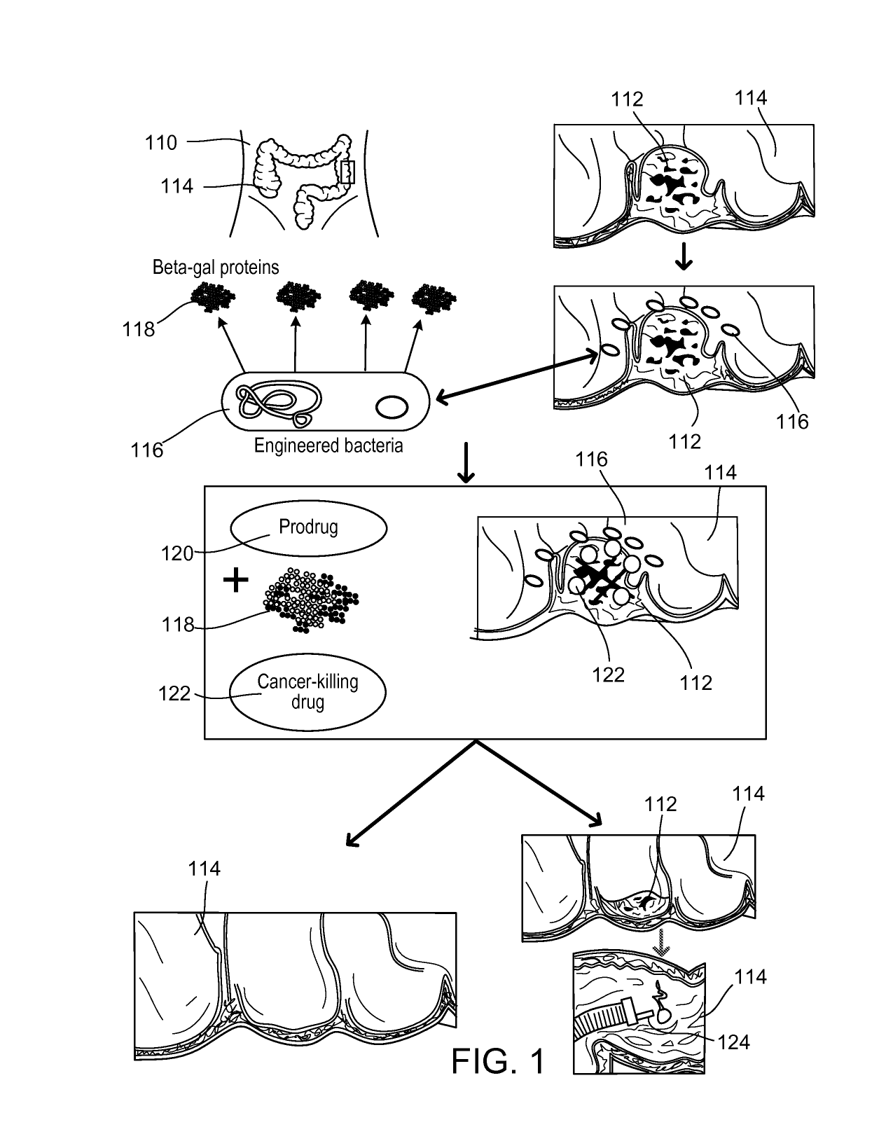

FIG. 1 illustrates an exemplary prodrug therapy for colon cancer that is useful in animals. In some embodiments, the animal is a human. In FIG. 1, an animal 110 having a tumor 112 in the large intestine 114 is provided with engineered bacteria 116. In this exemplary embodiment, the bacteria 116 have been engineered to express beta-gal protein 118. The engineered bacteria 116 can be provided, for example, orally in a liquid suspension, in a foodstuff, or as a pill or powder, or rectally as a suppository or a combination thereof. The engineered bacteria 116 colonizes around the tumor 112 and expresses the beta-gal protein 118. A prodrug 120 is provided to the animal 110 and is converted into an anti-cancer agent 122 in vivo. The action of the anti-cancer agent 122 may result in one of two outcomes shown. In the first outcome, the tumor 112 is no longer present in colon 114, in the second outcome, the tumor 112 has decreased in size and the tumor 112 in colon 114 which is now about the size of a small polyp can be removed without invasive surgery during a colonoscopy 124 procedure.

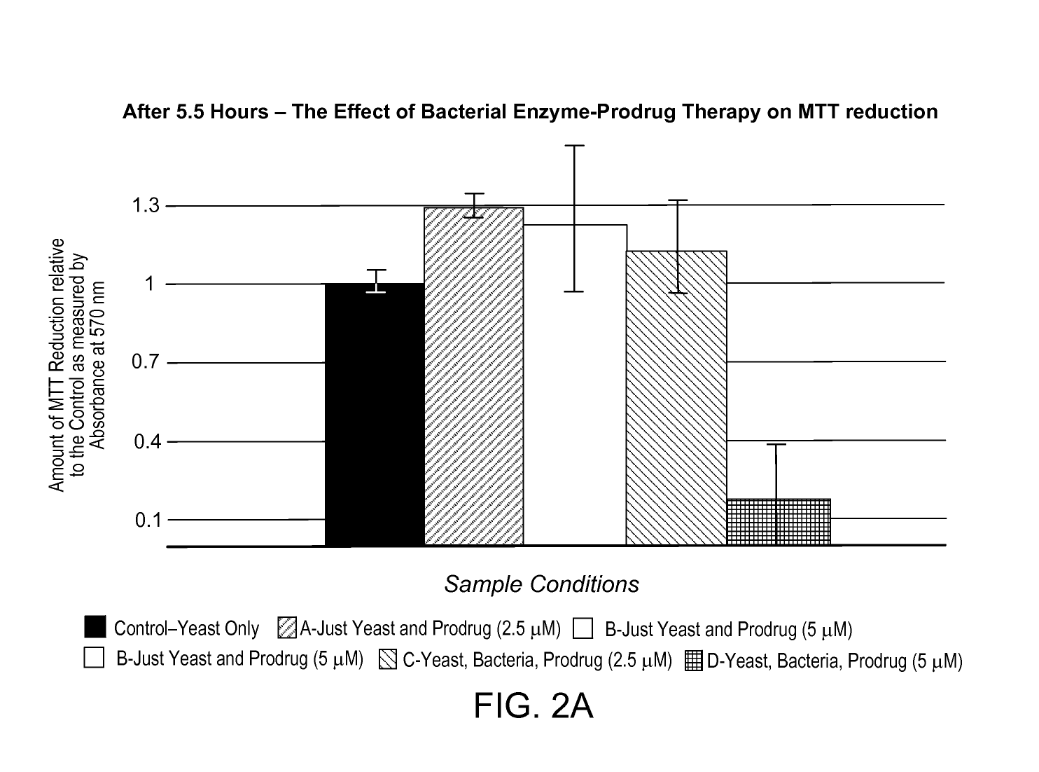

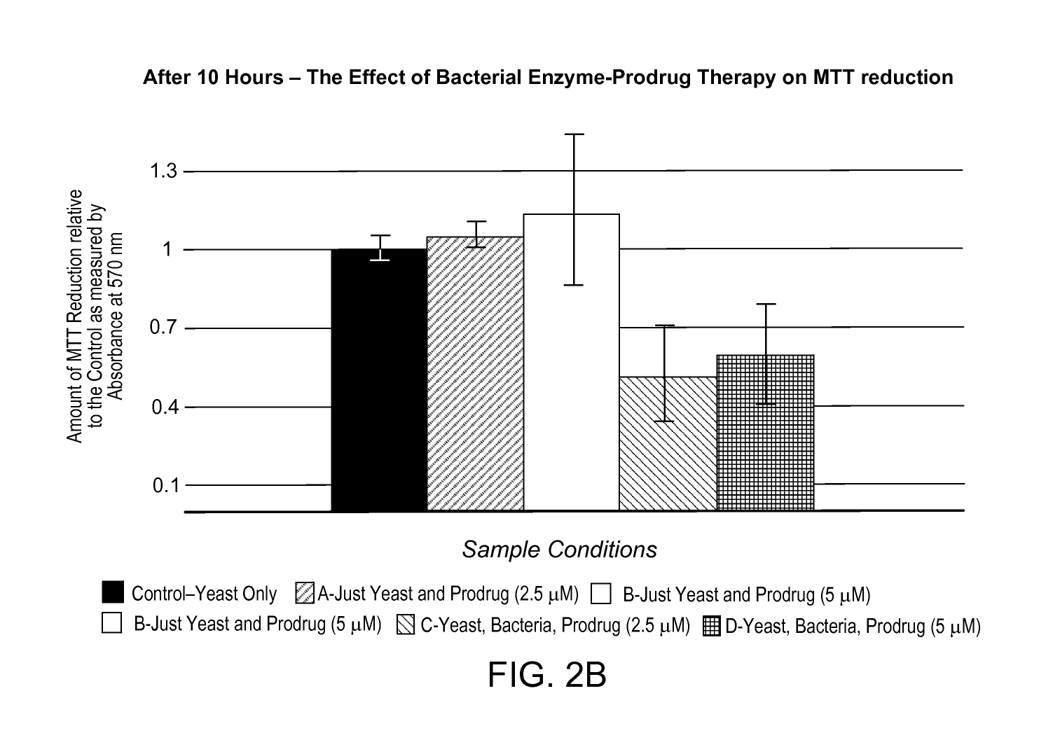

In an exemplary embodiment, E. coli BL21 cells were transformed with the pSV-β-Galactosidase plasmid which contains a gene for beta-gal (available from Promega, Wisconsin, USA). Other types of bacteria and differently configured plasmids can also be used. Yeast can act as a eukaryotic cell model for cancer cells. Yeast cells contain mitochondria and a number of proteins homologous to those in humans. A MTT assay was performed using the transformed E. coli, yeast cells, and Daun02 as the prodrug. MTT is a tetrazolium dye which is converted into an insoluble purple-colored product (formazan) by live cells. The presence of formazan was measured spectrophotometrically. Lower absorption values indicate yeast cell death. FIGS. 2A and 2B illustrate results of studies in which growing yeast cells were exposed to transformed E. coli BL21 cells and the prodrug Daun02. Bacteria and yeast were cultured over night and allowed to grow for 3 hours to be in optimal linear growth phase and samples were incubated together for approximately 5.5 or 10 hours in a 96 well microwell plate. The antibiotic kanamycin was added to all sample wells to reduce bacterial background signal. MTT dye was then added to wells and samples were incubated for three hours. A solubilization solution was then added, incubated for one hour, and the optical density was measured at 570 nm with a microplate reader. The absorbance at 570 nm of bacteria-only and bacteria and yeast-only samples were also measured. It was found that bacteria, by themselves, metabolize MTT dye to a certain extent and produce a background absorbance. This background absorbance was subtracted from samples C and D because the control sample and samples A and B did not contain any bacteria, and thus did not include any background absorbance. FIGS. 2A and 2B display results from different treatment times: 5.5 hours and 10 hours, respectively. Samples A and B had yeast and prodrug but did not have bacteria, and thus no added beta-gal enzyme. Samples A and B had absorption values close to the control values. Samples C and D contained beta-gal expressing bacteria, prodrug, and yeast cells. At 5.5 hours, Sample D had low absorption values demonstrating cell death by the treatment with 5 μM Daun02 and bacteria. At 10 hours, both Samples C and D exhibited yeast cell death. Sample C only had 2.5 μM Daun02 with bacteria, but at this time interval it showed effectiveness at the same level or even slightly greater than that of the Sample D which had a higher concentration. Results suggest that the effect of the treatment with the lower concentration of added prodrug increases with time. The length of time can be increased or decreased to increase the effectiveness of the treatment. Error bars demonstrating the average standard deviation of each sample set are shown. A 2-Sample T-Test between the control sample and Sample C in the 10 hour experiment returned a p-value of 0.00296. The 2-Sample T-Test between the control sample and Sample D after 10 hours returned a p-value of 0.00593. Both p-values are smaller than the alpha level of 0.05 chosen before experimentation. FIGS. 2A and 2B demonstrate a decrease in yeast model cell survival in samples treated with bacterial beta-gal enzyme-prodrug.

The effectiveness of the transformed bacteria for producing cell death in the presence of a prodrug was further investigated by fluorescent confocal and widefield microscopy. Calcofluor white M2R dye was used to selectively stain yeast chitin cell walls for fluorescent microscopy. The FUN-1 dye (Life Technologies, California, USA) was used to stain cylindrical yeast cells. If the yeast cell is alive, FUN-1 appears red, if the cell is dead, FUN-1 appears green. Only live yeast cells have red vacuolar structures indicating intact cell membranes. Thus the number of the vacuole structures as viewed under the red filter corresponds to the number of living yeast cells in a sample. The image analysis program CellC was utilized to count an approximate numbers of living cells for each image of the samples. The images from the fluorescent microscope demonstrated an 85% decrease in cell survival for the samples treated with transformed E. coli and prodrug as compared to the control.

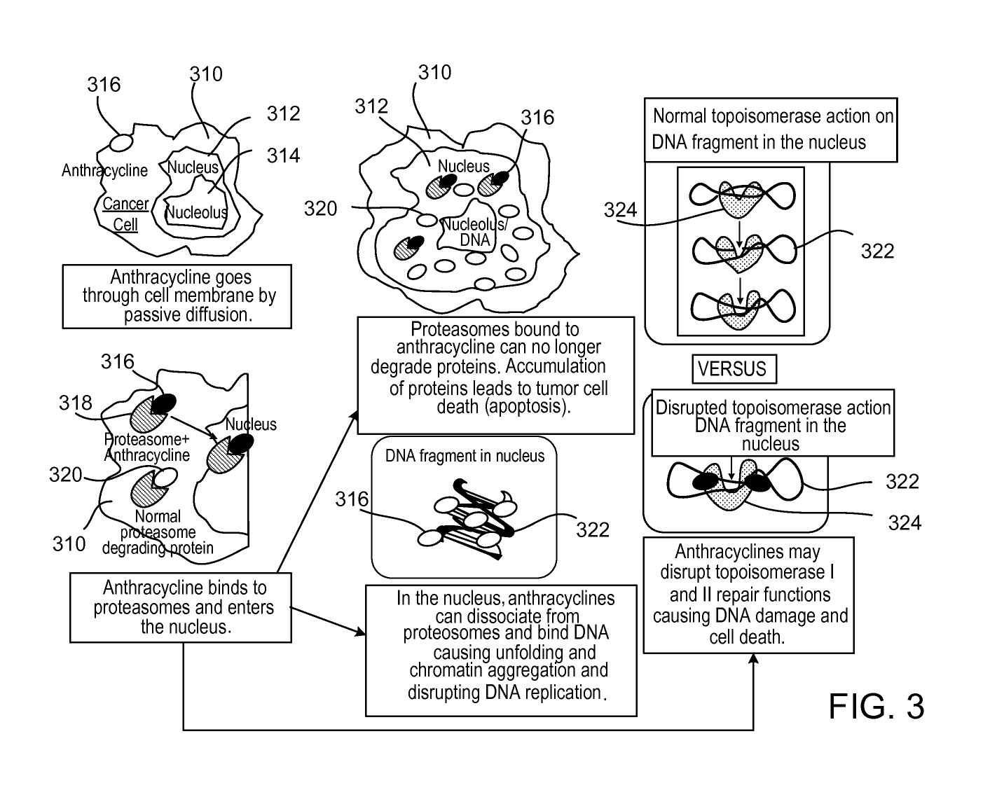

FIG. 3 schematically illustrates some possible mechanisms of action for anthracyclines on tumor cells leading to tumor cell death. In FIG. 3, a cancer cell 310 is shown having a nucleus 312 and a nucleolus 314. Anthracycline molecules 316 enter the cell 310 through passive diffusion. Once inside the tumor cell 310, the anthracycline 316 can bind to a proteasome 318 and enter the nucleus 312. A proteasome 318 having a bound anthracycline 316 is unable to bind and degrade proteins 320 normally. A buildup of protein in a cell leads to apoptosis and cell death. Additionally, once inside the nucleus, anthracycline molecules 316 can disassociate from the proteasome 318 and bind to DNA molecules 322. Anthracycline binding to DNA 322 leads to unfolding and chromatin aggregation which in turn inhibits DNA replication. Further, anthracyclines may disrupt the function of topoisomerase I and II 324 leading to DNA 322 damage and cell death.

The desired DNA construct for protein expression is produced through molecular biology techniques such as cloning and PCR. Selected bacteria are transformed with plasmids containing one or more therapeutic genes. Expression occurs either constitutively or by inducible promoter present on the plasmid and operatively coupled to the gene to be expressed. Proteins that can perform a therapeutic anti-cancer action such as inducing autophagy and/or apoptosis, having any other beneficial immunological, prodrug catalysis, anti-angiogenic, and/or other tumor suppressing ability are suitable for use. Other examples of beneficial proteins include protein 53 (p53) (cancer death via: interactions with numerous other pro-apoptotic proteins), HIV-Vpr (cancer death via: DNA double strand breaks), interleukins and other cytokines (cancer death via: interactions with other pro-appoptotic proteins, interactions with immune system, disruption of mitochondria, generation of reactive oxygen species, damage to mitochondria and/or the endoplasmic reticulum), endostatin (cancer death via: inhibition of angiogenesis), fragile histidine triad protein (cancer death via: evidence suggests the suppression of the oncogene HER2/neu and the synergizing with the Von Hippel-Lindau tumor suppressor), or tumor specific antigens (cancer death via: interactions with the immune system). The DNA in bacteria may also be engineered to be more tumor specific (as described herein, for example). Engineered DNA can be transformed into bacterial cells, such as, for example, E. coli, and bacteria from the genera: Lactococcus, Streptococcus, Clostridium, Salmonella, Listeria, Prevotella, Bifidobacterium, Leuconostoc, Peseudomonas, and Lactobacillus species (some of the listed bacterial species refer to deactivated strains that are non-virulent). Other examples of suitable bacteria species include: Streptococcus thermophilus, Lactobacillus bulgaricus, and Bifidobacterium lactis (BB-12). The engineered bacteria go through an incubation growth process. After having reached a stable growth rate they can be stored for later delivery, used in manufacturing of or added as an additive to a food product, or encapsulated in a probiotic tablet. For example the bacteria can be delivered orally in pill, powder, or suspension form in a liquid, a timed-release oral dose, by adding to a food base such as, for example, a cultured milk product such as yogurt, a cultured foodstuff, a foodstuff, or rectally as a suppository or a combination thereof. Cultured foodstuffs include probiotic beverages that may be dairy or non-dairy for example fermented oat drinks, altered fruit juices, or milk/yogurt-based beverages. Foodstuffs include, for example, cereal bars and chocolate.

Engineered bacteria are administered to an animal in need of treatment and one or more prodrugs (for example nucleoside or amino acid analogs, polymerase inhibitors, Daun02, CNOB, or other cancer-killing or apoptosis inducing molecules) is/are administered to the animal if the corresponding therapeutic protein has catalytic activity toward a prodrug. The prodrug(s) can be administered orally, in a therapeutic amount, in the form of, for example, a pill, a timed release capsule, in a foodstuff, a liquid solution or suspension, or as a powder, rectally as a suppository in solid or liquid form, injected into the patient in a solution or suspension, or a combination thereof. The therapeutic amount of the prodrug can be injected, for example into an artery leading to the cancer.

In additional embodiments, bacterially expressed proteins described herein can be shortened or otherwise modified versions of natural proteins. The proteins used in therapeutic treatment can have added sequences such as secretion tags, for example, Usp45 (a secretion tag), and be fused to human cell membrane penetrating protein like GST (glutathione S-transferase) which can enhance effectiveness of the therapeutic protein. Useful tags include, for example, Schistosoma japonicum-derived glutathione-S-transferase (GST)-tagged fusion protein. A secretion tag is not necessarily required for beta-galactosidase, however secretion tags can be used and include the hlyA or OmpA sequences. Therapeutic approaches can provide treatment, inhibitory, and/or preventative measures for colon cancers. Use of engineered bacteria that will pass through the digestive tract can be a practical and safe source of therapeutic and tumor inhibiting proteins.

In additional embodiments, transformed Lactococcus lactis is used in a tumor treatment. Lactococcus lactis is a gram positive bacteria used in the production of cheeses such as Colby, cheddar, cream, cottage, and blue cheese. It is also in buttermilk and fermented milk, sour cream, and various types of yogurt such as viili and filmjolk. Therefore, it is known to be safe and inexpensive. Using a nisin expression system, dosing of expressed proteins can be somewhat controlled. In additional embodiments a lactose promoter/repressor system can be used. With a lactose promoter/repressor expression system, the strength of treatment may be varied by controlling the lactose intake of the patient. Exemplary proteins that the Lactoccus lactis or another selected bacteria can secrete include proteins that are known to selectively kill tumor and colon cancer cells, that can be expressed in active therapeutic form by the bacterium, and that have low side-effects. Interleukin 24 (IL-24) also known as melanoma differentiation associated 7 (MDA-7) is an exemplary protein. It is selective to cancer and interacts with a variety of autophagy or apoptosis proteins. It has been shown to act through multiple cancer-killing pathways. Furthermore, it can act extracellularly and cause an anti-tumor bystander effect by autocrine signaling in which it binds to cell receptors and causes upregulation of its own expression. Intracellularly expressed proteins can then lead to autophagy or apoptosis of cells.

Bacteria such as Lactococcus lactis expressing a therapeutic protein such as, for example, IL-24, can inhibit colon cancer growth and metastasis through autocrine signaling mechanisms that cause further expression of the therapeutic protein itself; some as intracellular proteins, and its interactions with proteins leading to endoplasmic reticulum stress, ceramide-mediated stress, and generation of reactive oxygen species in cancer cells. The bacterially expressed therapeutic protein may also act through any of the previously described mechanisms.

For bacterial expression, the nisin controlled expression system is an example of an expression system useful in Lactococcus lactis. The nisin expression system allows secretion of proteins, is relatively easy to manipulate genetically because of its shuttle vector capability, and has expression dosing capabilities and well-defined transformation protocols. A subject treated with a bacterium that has been transformed with a plasmid bearing the nisin expression system, is also optionally administered nisin as part of a therapeutic regimen.

An exemplary gene sequence useful in a plasmid to transform Lactococcus lactis includes two NaeI restriction sites, a GST fusion protein sequence, a truncated IL-24 (tIL-24) sequence, and a stop codon:

<Seq. ID No. 1>
GCCGGCATTGGTCAAGTTGAAGATGTTGAATCAGAATATCATAAAACA
CTTATGAAACCACCAGAAGAAAAAGAAAAAATTTCAAAAGAAATTCT
TAATGGTAAAGTTCCAATTCTTCTTCAAGCTATTTGTGAAACACTTAAA
GAATCAACAGGTAATTTGACAGTTGGTGATAAAGTTACACTTGCTGAT
GTTGTTCTTATTGCTTCAATTGATCATATTACAGATCTTGATAAAGAAT
TTTTGACAGGTAAATATCCAGAAATTCATAAACATCGTAAACATCTTT
TGGCTACATCACCAAAACTTGCTAAATATCTTTCAGAACGTCATGCTA
CAGCTTTTTTTTCCATCAGAGACAGTGCACACAGGCGGTTTCTGCTATT
CCGGAGAGCATTCAAACAGTTGGACGTAGAAGCAGCTCTGACCAAAG
CCCTTGGGGAAGTGGACATTCTTCTGACCTGGATGCAGAAATTCTACA
AGCTCTAAGCCGGC

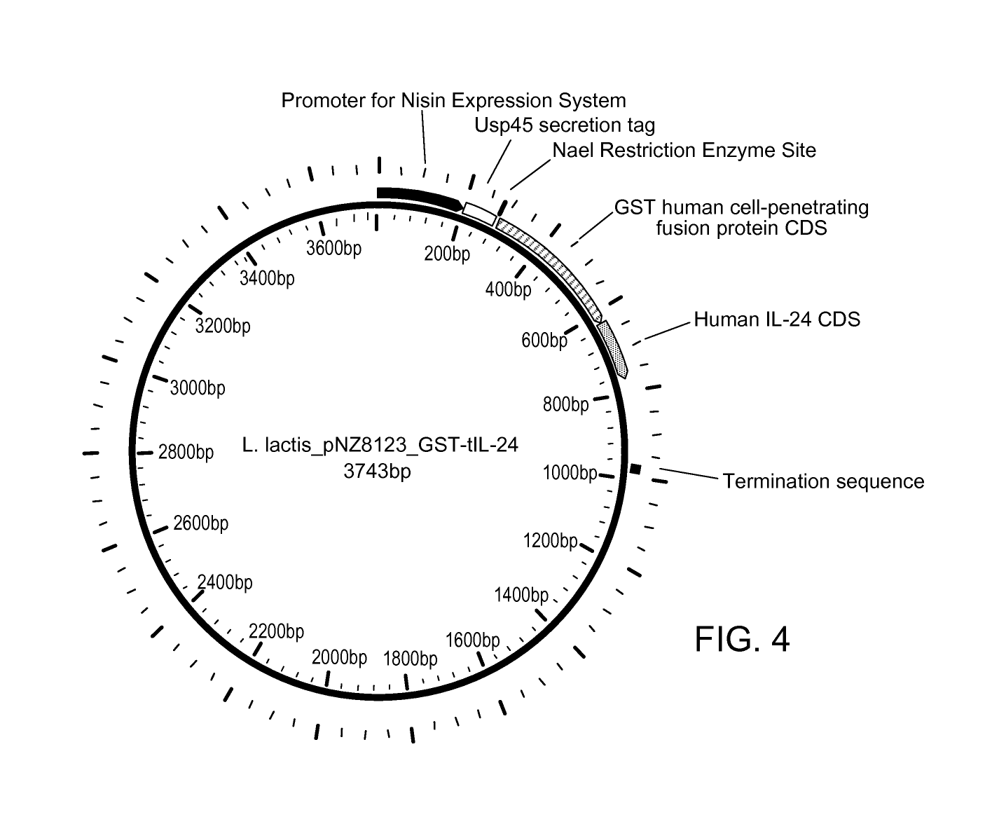

FIG. 4 provides a plasmid map for an exemplary plasmid that is useful to transform bacteria. The plasmid of FIG. 4 contains a promoter for the Nisin expression system, a Usp45 secretion tag, a GST human cell-penetrating fusion protein coding DNA sequence, a human IL-24 coding DNA sequence, and a termination sequence. Additionally, the plasmid contains a restriction enzyme site that allows for cloning the GST and IL-24 sequence into the plasmid backbone. Other plasmids are possible comprising, for example, different expression systems, different therapeutic proteins, different secretion tags (or no secretion tag), and different cell-penetrating proteins or peptides (or no cell penetrating protein).

Additional expression systems include a lactose promoter/repressor expression system. In this system, the strength of treatment may vary with amount of lactose administered to patient.

Provided are therapies and preventatives for cancers such as colon cancers that employ bacteria expressing therapeutic proteins that are capable of being added to foodstuffs, given as probiotic caplets or in liquid suspensions. Transformed bacterium according to certain embodiments can be optionally administered as either for therapeutic purposes or for preventative purposes. Additionally, more than one different type of bacteria can be used at one time and multiple prodrugs can be used at the same time.

Generally, treatment of tumors and malignant tumors includes slowing the growth of the tumor, inhibiting the spread of a tumor, inhibiting the spread of one or more metastases associated with a cancer, reducing the size of a tumor, and/or inhibiting the recurrence of cancer treated previously. Pharmaceutical compositions according to some embodiments can optionally include one or more pharmaceutically acceptable excipients.

Persons skilled in the relevant art appreciate that modifications and variations are possible throughout the disclosure as are substitutions for various components shown and described. Reference throughout this specification to “one embodiment” or “an embodiment” means that a particular feature, structure, material, or characteristic described in connection with the embodiment is included in at least one embodiment, but does not necessarily denote that they are present in every embodiment. Various additional elements may be included in some and/or described features may be omitted in other embodiments.

1. A method for inhibiting tumor growth in a subject in need thereof, comprising,
providing transformed bacteria comprising a plasmid wherein the plasmid codes for an protein wherein the protein is capable of catalyzing a reaction that converts a prodrug into a therapeutically active molecule to the gastrointestinal system of the subject,
and administering a prodrug to the subject.

2. The method of claim 1 wherein the tumor to be inhibited is adjacent to an intestine.

3. The method of claim 1 wherein the transformed bacteria are provided as a pill, a powder, a suspension in a liquid, a timed-release capsule, a foodstuff, or a cultured foodstuff.

4. The method of claim 1 wherein the transformed bacteria are selected from the group consisting of E. coli, Lactococcus, Streptococcus, Clostridium, Salmonella, Listeria, Bifidobacterium, and Lactobacillus.

5. The method of claim 1 wherein the transformed bacteria are selected from the group consisting of Streptococcus thermophilus, Lactobacillus bulgaricus, Bifidobacterium lactis (BB-12), and Prevotella.

6. The method of claim 1 wherein the transformed bacteria are E. coli.

7. The method of claim 1 wherein the transformed bacteria are Lactococcus lactis.

8. The method of claim 1 wherein the enzyme is selected from the group consisting of β-galactosidase, cytosine deaminase, glutathione S-transferase P1 (GSTP1), E. coli nitroreductase, herpes simplex 1 virus thymidine kinase, carboxypeptidase G2, purine nucleoside phosphorylase (PNP), nitrogen reductase, penicillin-V amidase, cytosine-deaminase (CD), and cytochrome P450 (CYP450).

9. The method of claim 1 wherein the prodrug is selected from the group consisting of Daun02, gal-DNC4,5-fluoro cytosine, (6-chloro-9-nitro-5-oxo-5H-benzo(a)phenoxazine), ganciclovir, (4 [(2-chloro ethyl)(2-mesyloxyethyl)amino]benzoyl 1-glutamic acid), 6-methylpurine deoxyriboside, (5-[aziridin-1-yl]-2,4-dinitrobenzamide), Irinotecan (CPT 11), 5-fluorocytosine, and cyclophosphamide.

10. The method of claim 1 wherein the prodrug is selected from the group consisting of nucleoside analogs, amino acid analogs, polymerase inhibitors, nitrogen mustard 1 glutamates, doxorubicins.

11. A method for inhibiting tumor growth in a subject in need thereof, comprising,
administering to the gastrointestinal system of the subject, a transformed bacteria comprising a plasmid wherein the plasmid comprises a gene for Interleukin 24 and the transformed bacteria are capable of excreting Interleukin 24.

12. The method of claim 11 wherein the transformed bacteria are selected from the group consisting of E. coli, Lactococcus, Streptococcus, Clostridium, Salmonella, Listeria, Bifidobacterium, and Lactobacillus.

13. The method of claim 11 wherein the transformed bacteria are Lactococcus.

14. The method of claim 13 wherein the plasmid additionally comprises a nisin-controlled expression system.

15. The method of claim 13 wherein the plasmid additionally comprises a lactose-controlled expression system.

16. The method of claim 11 wherein the plasmid additionally comprises a sequence coding for a secretion tag.

17. The method of claim 11 wherein the plasmid additionally comprises a sequence coding for a human cell membrane penetrating protein.

18. The method of claim 11 wherein the plasmid additionally comprises a sequence coding for glutathione S-transferase.

19. The method of claim 11, wherein the gene for Interleukin 24 comprises a gene coding for any isoform of the Interleukin 24 protein or any part thereof, including any truncated, condon-optimized, or alternatively spliced isoforms of the gene.
